(19) United States
(12) Patent Application Publication
(10) Pub. No.: US 2020/0316019 A1
(43) Pub. Date: Oct. 8, 2020

(54) METHOD FOR TREATING PROTEIN AGGREGATION DISEASES

(71) Applicant: NATIONAL TAIWAN NORMAL UNIVERSITY, Taipei City (TW)

(72) Inventors: Guey-Jen LEE-CHEN, Taipei City (TW); Wenwei LIN, Taipei City (TW); Ya-Jen CHIU, Taipei City (TW); Te-Hsien LIN, Taipei City (TW)

(73) Assignee: NATIONAL TAIWAN NORMAL UNIVERSITY, Taipei City (TW)

(21) Appl. No.: 16/910,786

(22) Filed: Jun. 24, 2020

Related U.S. Application Data
(63) Continuation-in-part of application No. 15/655,967, filed on Jul. 21, 2017, now Pat. No. 10,716,768.

(30) Foreign Application Priority Data
Aug. 11, 2016 (TW) 105125668

(51) Int. Cl. A61K 31/366; A61P 25/28

(52) U.S. Cl. CPC A61K 31/366; A61P 25/28

(57) ABSTRACT

The present invention provides a method for treating a protein aggregation disease, including: administering a pharmaceutical composition comprising a compound represented by the following formula (I) to a subject in need thereof,

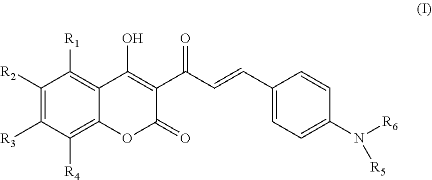

wherein each R₁, R₂, R₃, R₄, R₅ and R₆ are defined in the specification.

### CROSS REFERENCE TO RELATED APPLICATION

This application is a continuation-in-part (CIP) of U.S. patent application for “Method for Treating Abnormal β-Amyloid Aggregation Mediated Diseases”, U.S. application Ser. No. 15/655,967 filed Jul. 21, 2017, and the subject matter of which is incorporated herein by reference.

This application claims the benefits of the Taiwan Patent Application Serial Number 105125668, filed on Aug. 11, 2016, the subject matter of which is incorporated herein by reference.

### BACKGROUND OF THE INVENTION

### 1. Field of the Invention

The present invention relates to a method for treating protein aggregation diseases with a pharmaceutical composition.

### 2. Description of Related Art

Abnormal protein aggregation or inclusion is present in most age-related neurodegenerative diseases, wherein β-amyloid (Aβ) aggregation, tau protein aggregation, and α-synuclein aggregation are common protein aggregations. Furthermore, β-amyloid aggregation or tau protein aggregation causes Alzheimer's disease (AD), and α-synuclein aggregation causes Parkinson's disease.

Alzheimer's disease is the most well-known form of dementia and it causes memory loss and progressive cognitive decline. However, there is no drug for curing Alzheimer's disease at the moment, and the existing treatments for AD can merely preserve or improve cognitive function and reduce behavioral disorders to delay disease progression. As a result, there is an urgent need to find methods for treating Alzheimer's disease.

Pathological features of Alzheimer's disease include extracellular amyloid and intracellular neurofibrillary tangle, wherein the main component of extracellular amyloid is β-amyloid and the intracellular neurofibrillary tangle is composed of excessively phosphorylated tau protein. Furthermore, abnormal β-amyloid and tau protein depositions increase oxidative stress and thus lead to death of nerve cells. Hence, the level of deposition is strongly relevant to neurotoxicity.

Parkinson's disease (PD) is the second most common neurodegenerative disorder affecting 1% people older than 60 years old. The symptoms commonly seen in PD patients are resting tremor, rigidity, bradykinesia and postural instability. Pathologically, PD is defined by the presence of α-synuclein-containing Lewy bodies and Lewy neuritis in the subcortical regions of the brain. The α-synuclein tends to form oligomers, fibrils and aggregates, which have been considered the culprits to cause neurotoxicity.

Therefore, it is desirable to provide a compound to inhibit abnormal β-amyloid, tau protein, or α-synuclein aggregation, and the compound can be used to treat or effectively delay neurodegenerative diseases related to abnormal protein aggregation such as Alzheimer's disease and Parkinson's disease.

### SUMMARY OF THE INVENTION

One object of the present invention is to provide a pharmaceutical composition comprising a compound represented by the following formula (I):

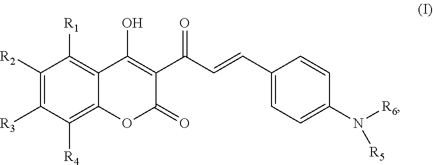

wherein each R₁, R₂, R₃, R₄, R₅ and R₆ is independently selected from the group consisting of H and alkyl.

Another object of the present invention is to provide a method for treating or delaying protein aggregation diseases, for example, Alzheimer's disease or Parkinson's disease.

To achieve the object, the present invention provides a method for treating or delaying a protein aggregation disease, comprising: administering the aforementioned pharmaceutical composition to a subject in need thereof.

Another object of the present invention is to provide a method for inhibiting protein aggregations in a subject.

To achieve the object, the present invention provides a method for inhibiting a protein aggregation in a subject in need thereof, comprising: administering the aforementioned pharmaceutical composition to the subject.

In the present invention, the protein aggregation disease may be a protein aggregation mediated disease, such as Alzheimer's disease or Parkinson's disease. In one aspect of the present invention, the protein aggregation disease is Alzheimer's disease. In another aspect of the present invention, the protein aggregation disease is Parkinson's disease.

In one aspect of the present invention, the protein aggregation may be β-amyloid aggregation, tau protein aggregation, or α-synuclein aggregation.

Another object of the present invention is to provide a method for inhibiting inflammation in a subject.

To achieve the object, the present invention provides a method for inhibiting inflammation in a subject in need thereof, comprising: administering the aforementioned pharmaceutical composition to the subject.

Yet another object of the present invention is to provide a method for inhibiting pro-inflammatory mediators, pro-inflammatory cytokines, or increasing nerves protection in a subject.

To achieve the object, the present invention provides a method for inhibiting pro-inflammatory mediators, pro-inflammatory cytokines, or increasing nerves protection in a subject in need thereof, comprising: administering the aforementioned pharmaceutical composition to the subject.

In the present invention, the pro-inflammatory mediators may be any pro-inflammatory mediators. Preferably, the pro-inflammatory mediator is NO.

In the present invention, the pro-inflammatory cytokines may be any pro-inflammatory cytokines. Preferably, the pro-inflammatory cytokine is IL-1β, IL-6 or TNF-α.

In the present invention, increasing nerves protection may be achieved by any means. Preferably, increasing nerves protection may be achieved by promoting neurite outgrowth.

In the present invention, each R₁, R₂, R₃, and R₄ may be independently selected from the group consisting of H and alkyl. In one aspect of the present invention, each R₁, R₂, R₃, and R₄ may be independently selected from the group consisting of H and C1-C5 alkyl. Preferably, R₁, R₂, R₃, and R₄ is H.

In the present invention, R₅ may be H or alkyl. In one aspect of the present invention, R₅ is C1-C5 alkyl. Preferably, R₅ is methyl or ethyl. More preferably, R₅ is methyl.

In the present invention, R₆ may be H or alkyl. In one aspect of the present invention, R₆ is C1-C5 alkyl. Preferably, R₆ is methyl or ethyl. More preferably, R₆ is methyl.

In another aspect of the present invention, the compound represented by formula (I) may be a compound represented by the following formula (I-1):

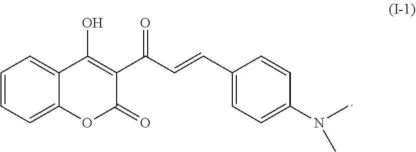 [SMILES: CN(C)c1ccc(/C=C/C(=O)c2c(O)c3ccccc3oc2=O)cc1]

However, the compound represented by formula (I) is not limited to the aforementioned compound represented by formula (I-1).

In the pharmaceutical composition of the present invention, the concentration of the aforementioned compounds, such as compounds of formula (I) or formula (I-1) is not particularly limited and can be adjusted according to actual use such as route of administration, the carrier, the diluent, the excipient, complementary medicines, disorder severity, and the like. A person skilled in the art can adjust the dose to obtain desired curative effect.

In one aspect of the present invention, the concentration of the compound is in a range from 0.0005 μM to 200 μM, preferably 5 μM to 180 μM, more preferably 10 μM to 160 μM, based on a total weight of the pharmaceutical composition.

In one aspect of the present invention, the pharmaceutical compositions prepared by the present invention may include one or more any compounds described above.

In one aspect of the present invention, the pharmaceutical composition may further comprise: at least one pharmaceutically accepted carrier, a diluent, or an excipient. The carrier, the diluent, or the diluent must be acceptable, which means they are compatible with main ingredients of the pharmaceutical composition (preferably capable of stabilizing the main ingredient), and are not harmful to the target individuals. For example, the compound can be encapsulated into liposome to facilitate delivery and absorption. Alternatively, the compound can be diluted with aqueous suspension, dispersion or solution to facilitate injection. Or, the compound can be produced in a form of a capsule or tablet for storage and carrying.

In one aspect of the present invention, the prepared pharmaceutical composition of the present invention can be formulated in a solid or liquid form. When the pharmaceutical composition is in the solid form, solid excipients may include powders, granules, tablets, capsules suppositories, and the like. Pharmaceutical compositions in solid form may further include, but not limited to, solid formulation such as flavoring agents, binders, preservatives, disintegration agents, glidants, and the like. In addition, the liquid excipients used in the pharmaceutical compositions in liquid form may include water, solutions, suspensions, emulsion, and the like. The pharmaceutical compositions in liquid form may further include liquid formulations, for example, but not limited to, coloring agents, flavoring agents, dispersing agents, antibacterial agents, stabilizers, viscosity-increasing agents, and the like.

For example, a powder formulation may be prepared by simply mixing the compound used in the present invention with suitable pharmaceutically acceptable excipients such as sucrose, starch and microcrystalline cellulose. A granule formulation may be prepared by mixing the compound used in the present invention with suitable pharmaceutically acceptable excipients, and suitable pharmaceutically acceptable binders such as polyvinyl pyrrolidone and hydroxypropyl cellulose, followed by wet granulation method using a solvent, such as water, ethanol, and isopropanol, or followed by dry granulation method using compression force. In addition, a tablet formulation may be prepared by mixing the granule formulation with suitable pharmaceutically acceptable glidants, such as magnesium stearate, followed by tableting using a tablet machine. Hence, a person skilled in the art can appropriately choose suitable formulation to meet the needs.

To implement the method according to the present invention, the above pharmaceutical composition can be administered via parenteral administering, oral administering, nasal administering, rectal administering, topical administering, or sublingual administering.

The term “parenteral” used herein refers to subcutaneous injection, intradermal injection, intravenous injection, intramuscular injection, intra-articular injection, intraocular injection, intrasternal injection, cerebrospinal injection, intra-lobular or intracranial injection, and any other suitable injection technique. The pharmaceutical compositions for oral administration may be in any formulation acceptable for oral administration including granules, capsules, tablets, emulsions, aqueous suspensions, dispersing agents, and solutions.

The term “treat” used herein refers to achieving the desired medical and physical effect. The effect may be preventing or partially preventing a disease, a prophylactic method for a symptom or condition thereof, completely or partially cure of disease, or a therapy for symptoms or adverse reactions caused by a disease. The term “treatment” used herein encompasses the treatment for mammals, especially for human diseases, and includes prevention of a disease which is predisposed but not diagnosed, or alleviating a disease which means a use for alleviating a disease and/or the symptoms thereof.

The term “delay”, “inhibit”, “decrease”, or “reduce” used herein refers to a case that the pharmaceutical composition of the present invention is applied to a subject suffering from a protein aggregation disease, having symptoms or disorder of the disease, or having a tendency of development of the disease in order to achieve cure, mitigation, alleviation, improvement, or recovery of the tendency of the symptoms.

Other objects, advantages, and novel features of the present invention will become more apparent from the following detailed description when taken in conjunction with the accompanying drawings.

### BRIEF DESCRIPTION OF THE DRAWINGS

FIG. 1 is a graph showing the comparative radical scavenging activity of kaempferol and compound (I-1) on DPPH according to one embodiment of the present invention.

FIGS. 2A-2D respectively show secretion analyses of NO, IL-1β, IL-6, and TNF-α in α-synuclein fibrils (2 μM)-activated BV-2 microglial cells treated with or without the compound (I-1) according to one embodiment of the present invention.

FIG. 3A and FIG. 3B are graphs showing the compound (I-1)-mediated kinase activation in CRE-GFP reporter cells according to one embodiment of the present invention.

FIG. 4A are western blot images showing the levels of p-CREB, CREB, pro-BDNF, and m-BDNF in immunoblot according to one embodiment of the present invention; and FIG. 4B are graphs showing the quantified results of FIG. 4A according to one embodiment of the present invention.

FIG. 5A are graphs showing the neurite total length, process and branch in Aβ-GFP SH-SY5Y cells with or without the treatment of the compound (I-1) according to one embodiment of the present invention; and FIG. 5B are graphs showing the neurite total length, process and branch in ΔK280 tau_{RD}-DsRed-expressing SH-SY5Y cells with or without the treatment of the compound (I-1) according to one embodiment of the present invention.

### DETAILED DESCRIPTION OF THE PREFERRED EMBODIMENT

The accumulation of misfolded and aggregated proteins is one of the common pathological hallmarks of neurodegenerative diseases. These abnormal protein depositions lead to neurodegeneration via a number of mechanisms, including increased reactive oxygen species and associated neuroinflammation, and down-regulation of cAMP-response-element (CRE) binding protein 1 (CREB) signaling pathway. We examined the compound of the present invention as a therapeutic strategy for aggregation-prone neurodegenerative diseases.

[Compounds]

Please refer to Table 1. Table 1 shows the structure, chemical formula, and molecular weight of compounds tested in the following experiments, namely compound (I-1). However, the test compounds are not limited to the compound described below.

TABLE 1

| | | | Molecular |
|---|---|---|---|
| | | Chemical | Weight |
| Compound | Structure | Formula | (g/mole) |
| Compound (I-1) | 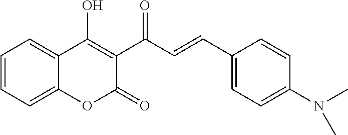 [SMILES: CN(C)c1ccc(/C=C/C(=O)c2c(O)c3ccccc3oc2=O)cc1] | C₂₀H₁₇O₄ | 335.35 |

[Solubility Test for Compounds]

This experiment was to test the solubility of compound (I-1) in cell culture medium. After vortex mixing and centrifugation for five minutes at 13,000 g, the compounds were completely soluble in the cell culture medium up to 200 μM.

[1,1-diphenyl-2-picryl hydrazyl (DPPH) Assay]

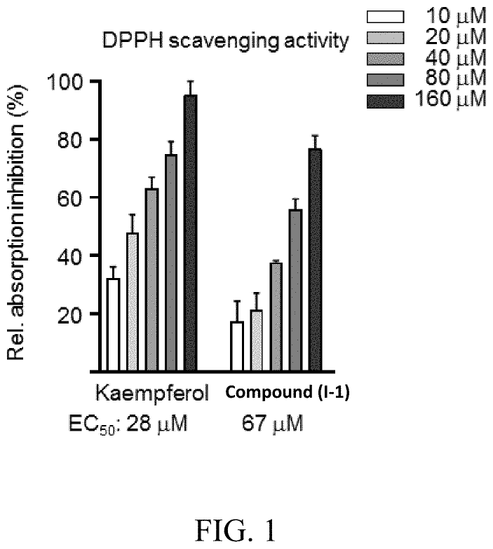

FIG. 1 is a graph showing the comparative radical scavenging activity of kaempferol and compound (I-1) on DPPH.

The free radical scavenging activities of tested compound (I-1) and kaempferol (as a positive control) were determined using the stable 1,1-diphenyl-2-picrylhydrazyl (DPPH, Sigma) free radical assay (see Li, N.; Liu, J. H.; Zhang, J.; Yu, B. Y. Comparative evaluation of cytotoxicity and antioxidative activity of 20 flavonoids. Journal of Agricultural and Food Chemistry 56:3876-3883; 2008) with some modifications. Briefly, radical scavenging activity was measured in an ethanol mixture containing 100 μM DPPH radical solution and the tested compound (10-160 μM) or kaempferol (10-160 μM). The mixture was vortexed for 15 sec and then left to stand at room temperature for 30 min. Then, the scavenging capacity was measured by monitoring the decrease in absorbance at 517 nm by a Thermo Scientific Multiskan GO Microplate Spectrophotometer. The radical scavenging activity was calculated using the formula: 1−(absorbance of sample/absorbance of control)×100%. The antioxidative activity expressed as EC₅₀ was defined as the concentration of the compounds required for inhibition of the formation of DPPH radicals by 50%.

The DPPH radical is a stable organic radical with an absorption band in 517 nm. Kaempferol, a natural flavonol with strong antioxidant property, was chosen as the reference antioxidant for this test. The EC₅₀ values of the DPPH scavenging activity were calculated. As shown in FIG. 1, DPPH scavenging activity was seen in the compound (I-1) with EC₅₀ being 67 μM. Therefore, the compound (I-1) has radical scavenging activity, and thus it can be used to treat Alzheimer's disease or Parkinson's disease.

[Anti-Inflammatory Activity Test]

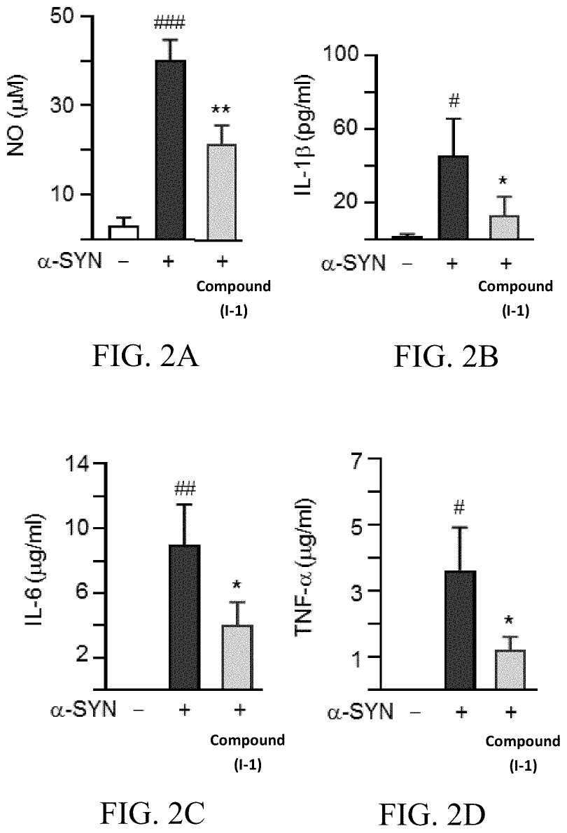

FIGS. 2A-2D respectively show secretion analyses of NO, IL-1β, L-6, and TNF-α in α-synuclein fibrils (2 μM)-activated BV-2 microglial cells treated with or without the compound (I-1).

Anti-inflammatory activity of compound (I-1) on α-synuclein fibrils-activated BV-2 cells was examined in this embodiment. BV-2 cells were plated in 1% FBS on day 1. After 20 hours, the cells were pretreated with compound (I-1) for 8 hours, followed by α-synuclein fibrils (α-Syn, 2 μM) treatment. After 20 hours, the cells were examined for NO release and IL-1β, IL-6 and TNF-α production. The results were analyzed with two-tailed Student's t test, wherein p values: comparisons between with and without fibril addition (^{#}: p<0.05, ^{##}: p<0.01, ^{###}: p<0.001), or between with and without compound (I-1) treatment (*: p<0.05, **: p<0.01).

As shown in FIG. 2A to FIG. 2D, treatment with compound (I-1) (10 μM) significantly reduced nitric oxide (NO) (from 40.1 μM to 21.7 μM, p=0.001), interleukin (IL)-1β (from 45.8 to 12.9 pg/ml, p=0.038), IL-6 (from 9.0 to 4.0 μg/ml, p=0.019) and tumor necrosis factor (TNF)-α (from 3.6 to 1.2 μg/ml, p=0.029).

[Compound (I-1) Enhancing CRE-Mediated Gene Expression Through PKA, CaMKII and ERK Kinases]

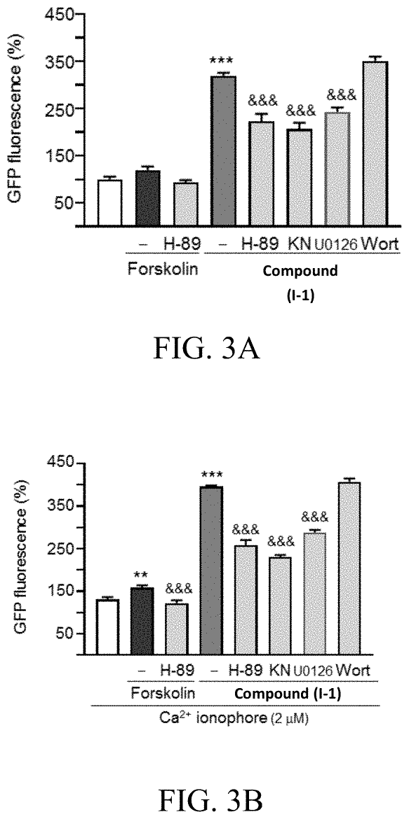

FIG. 3A and FIG. 3B are graphs showing the compound (I-1)-mediated kinase activation in CRE-GFP reporter cells.

Flp-In 293 GFP reporter cells with GFP reporter driven by the cAMP response element (CRE) motifs-TATA-like promoter was established to examine the mechanism of compound (I-1) mediating activation of CREB. Firstly, seed the cells and wait for 20 hours. Then, kinase inhibitor H-89 (PKA inhibitor), KN-62 (CaMKII inhibitor), U0126 (ERK inhibitor) or wortmannin (PI3K inhibitor) (10 μM) was added to CRE-GFP reporter cells for 4 hours, followed by the addition of compound (I-1) (10 μM) and/or Ca²⁺ ionophore (2 μM) for 5 hours. Forskolin, an activator of adenylyl cyclase, was included as a control for comparison.

GFP fluorescence level was assessed by flow cytometry (n=3). Results were analyzed with one-way ANOVA with a post hoc Tukey test. To normalize, GFP fluorescence level in untreated cells was set at 100%. p values: comparisons between compound treated vs. untreated cells (**: p<0.01, ***: p<0.001) or kinase inhibitor-treated vs. untreated cells (^{&&&}: P<0.001).

As shown in FIG. 3A and FIG. 3B, compound (I-1) significantly activated CRE-mediated transcription in the presence (from 131% to 395%, p<0.001) or absence (from 131% to 320%, p<0.001) of Ca²⁺ ionophore. Among the kinase inhibitors tested, H-89, KN-62, U0126, but not wortmannin, attenuated the GFP levels in the presence (258-288%, p<0.001) or absence (222-244%, p<0.001) of Ca²⁺ ionophore. Therefore, the results indicated that compound enhanced CRE-mediated gene expression through PKA, CaMKII and ERK kinases.

[Compound (I-1)-Mediated Kinase Activation in Aβ-GFP-Expressing SH-SY5Y Cells]

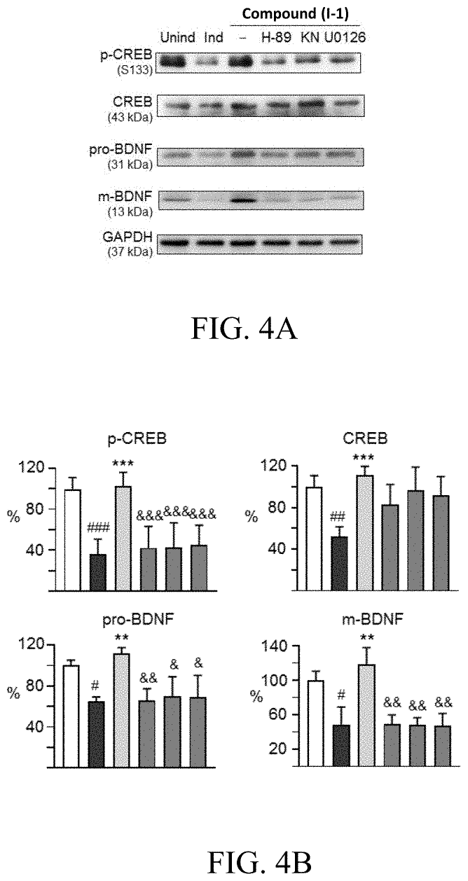

FIG. 4A are western blot images showing the levels of p-CREB, CREB, pro-BDNF, and m-BDNF in immunoblot; and FIG. 4B are graphs showing the quantified results of FIG. 4A.

The cAMP-response element binding protein (CREB) is a protein regulating the expression of genes that are important in dopaminergic neurons. Neurotrophins, such as brain derived neurotrophic factor (BDNF), are critical regulators during neurodevelopment and synaptic plasticity. The CREB is one of the major regulators of neurotrophin responses since phosphorylated CREB binds to a specific sequence in the promoter of BDNF and regulates its transcription. Following phosphorylation at Ser133, CREB upregulates BDNF expression to modulate synaptic activity.

To examine the CREB-mediated neuroprotective potential of compound (I-1), we applied kinase inhibitor H-89, KN-62 or U0126 to compound (I-1)-treated Aβ-GFP-expressing SH-SY5Y cells. On day 1, cells were plated with retinoic acid (RA, 10 μM) added to the culture medium. On day 2, compound (I-1) (5 μM) was added to the cells for 8 hours, followed by inducing Aβ-GFP expression with doxycycline (Dox, 5 μg/ml). Kinase inhibitors (10 μM) were added to the cells on day 6. On day 8, CREB and BDNF levels were measured by immunoblot using GAPDH (glyceraldehyde-3-phosphate dehydrogenase) as a loading control (n=3). The results obtained by immunoblot was quantified and analyzed with one-way ANOVA with a post hoc Tukey test. To normalize, protein expression level in untreated cells was set at 100%. p values: comparisons between induced vs. uninduced cells (^{#}: p<0.05, ^{##}: p<0.01, ^{###}: p<0.001), compound (I-1)-treated vs. untreated cells (**: p<0.01, ***: p<0.001), or kinase inhibitor-treated vs. untreated cells (^{&}: P<0.05, ^{&&}: P<0.01, ^{&&&}: P<0.001).

As shown in FIG. 4B, induced expression of Aβ-GFP reduced p-CREB (36% of control, p<0.001) and CREB (52% of control, p=0.003), as well as precursor (pro-BDNF) (65% of control, p=0.038) and mature (m-BDNF) (48% of control, p=0.011) BDNF protein levels, and treatment with compound (I-1) significantly increased p-CREB (from 36% to 103%, p<0.001), CREB (from 52% to 111%, p<0.001), pro-BDNF (from 65% to 111%, p=0.006), and m-BDNF (from 48% to 119%, p=0.001), whereas H-89, KN-62 or U0126 treatment mitigated the increase of p-CREB (from 103% to 45-43%, p<0.001), pro-BDNF (from 111% to 70-66%, p=0.013-0.007) and m-BDNF (from 119% to 49-47%, p=0.001).

The results indicated that compound (I-1) can upregulate brain-derived neurotrophic factor (BDNF) expression to modulate synaptic activity through CREB, which is in favour of synaptic activity.

[Neurite Outgrowth-Promoting Effect of Compound (I-1) in Aβ-GFP and ΔK280 tau_{RD}-DsRed-expressing SH-SY5Y cells]

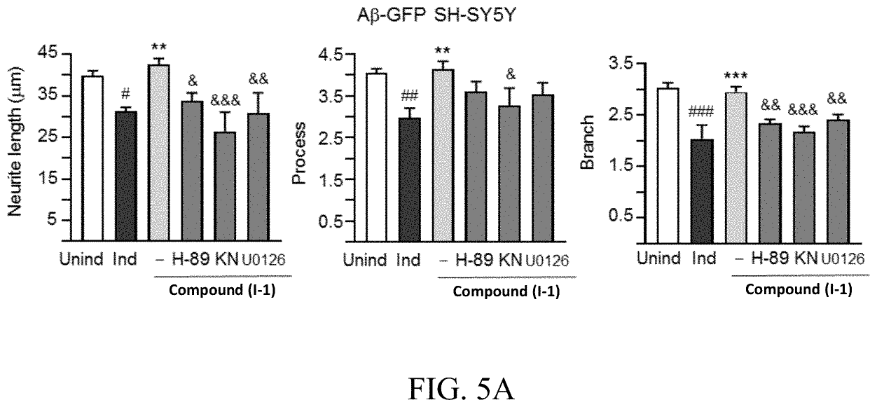

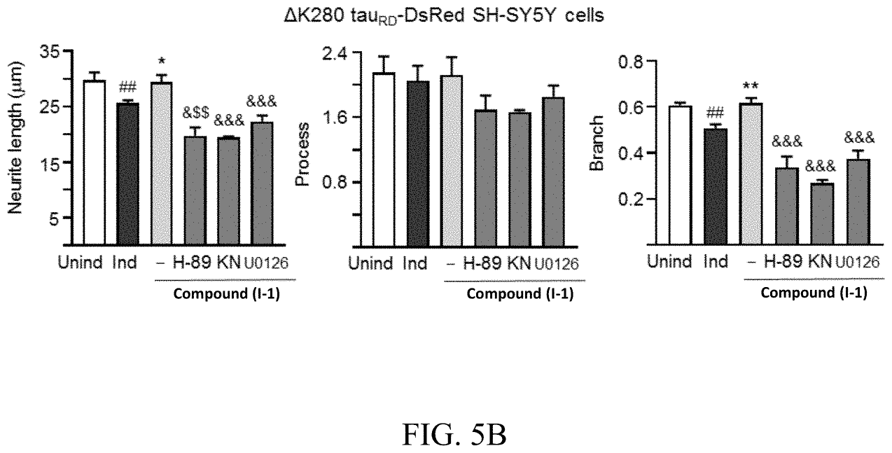

FIG. 5A are graphs showing the neurite total length, process and branch in Aβ-GFP SH-SY5Y cells with or without the treatment of the compound (I-1); and FIG. 5B are graphs showing the neurite total length, process and branch in ΔK280 tau_{RD}-DsRed-expressing SH-SY5Y cells with or without the treatment of the compound (IA).

Tet-On Aβ-GFP and ΔK280 tau_{RD}-DsRed SH-SY5Y cells were used to examine the neurite outgrowth-promoting potential of compound (I-1). As described, cells were plated with retinoic acid addition on day 1, compound (I-1) addition (5 μM in Aβ-GFP cells or 10 μM in ΔK280 tau_{RD}-DsRed cells) on day 2 for 8 hours, followed by inducing Aβ-GFP/ΔK280 tau_{RD}-DsRed expression with doxycycline (5 or 2 μg/ml). Kinase inhibitors (10 μM) were added to the cells on day 6. On day 8, the cells were fixed (4% paraformaldehyde for 15 min), permeabilized (0.1% Triton X-100 for 10 min), blocked (3% BSA for 20 min), and stained with TUBB3 (neuronal class III β-tubulin) primary antibody at 4° C. overnight, followed by fluorescent secondary antibody at room temperature for 3 hours. Neuronal images were captured and neurite outgrowth (length, process, branch) and analyzed (n=3). p values: comparisons between induced vs. uninduced cells (^{#}: P<0.05, ^{##}: P<0.01, ^{###}: P<0.001), compound (I-1)-treated vs. untreated cells (*: P<0.05, **: P<0.01, ***: P<0.001), or kinase inhibitor-treated vs. untreated cells (^{&}: P<0.05, ^{&&}: P<0.01, ^{&&&}: P<0.001). (one-way ANOVA with a post hoc Tukey test)

As shown in FIG. 5A, in Aβ-GFP-expressing SH-SY5Y cells, compound (I-1) rescued the reduced neurite total length (from 31.2 to 42.5 μm, p=0.006), process (from 2.97 to 4.15, p=0.002) and branch (from 2.04 to 2.95, p<0.001), whereas H-89, KN-62 or U0126 treatment counteracted the improvement (length: from 42.5 to 33.8-26.5 μm, p=0.034-<0.001; process: from 4.15 to 3.60-3.26, p=0.179-0.013; branch: from 2.95 to 2.41-2.18, p=0.005-<0.001). As shown in FIG. 5B, in ΔK280 tau_{RD}-DsRed-expressing SH-SY5Y cells, compound (I-1) rescued the reduced neurite total length (from 25.7 to 29.4 μm, p=0.013) and branch (from 0.51 to 0.62, p=0.004), whereas H-89, KN-62 or U0126 treatment counteracted the improvement (length: from 29.4 to 22.3-19.4 μm, p<0.001; branch: from 0.62 to 0.38-0.26, p<0.001).

The results shown in FIG. 5A and FIG. 5B indicate that the compound (I-1) can promote neurite outgrowth and protect nerves.

The compound of the present invention such as compound (I-1) exhibited free radical scavenging activity by DPPH biochemical assay and anti-inflammatory potential by reducing the expression and release of pro-inflammatory mediators in α-synuclein-stimulated mouse BV-2 microglial cells. In addition, compound (I-1) displayed potential to enhance CREB-mediated gene expression through protein kinase A (PKA), Ca²⁺/calmodulin dependent protein kinase II (CaMKII) and extracellular signal-regulated kinase (ERK) in CRE-GFP reporter cells. In Aβ-GFP or ΔK280 tau_{RD}-DsRed-expressing SH-SY5Y cells, compound (I-1) up-regulated CREB phosphorylation and downstream BDNF gene, and promoted neurite outgrowth, whereas blockage of PKA, CaMKII or ERK pathway counteracted the beneficial effects of compound (I-1). Therefore, the compound of the present invention can be used to treat Alzheimer's disease or Parkinson's disease.

Although the present invention has been explained in relation to its preferred embodiment, it is to be understood that many other possible modifications and variations can be made without departing from the spirit and scope of the invention as hereinafter claimed.

What is claimed is:

1. A method for treating a protein aggregation disease, comprising:
administering a pharmaceutical composition comprising a compound represented by the following formula (I) to a subject in need thereof, 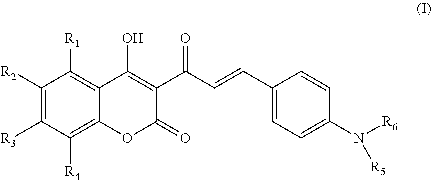
wherein each R₁, R₂, R₃, R₄, R₅ and R₆ is independently selected from the group consisting of H and alkyl.

2. The method of claim 1, wherein each R₁, R₂, R₃, R₄, R₅ and R₆ is independently selected from the group consisting of H and C1-C5 alkyl.

3. The method of claim 1, wherein each R₁, R₂, R₃, and R₄ is H.

4. The method of claim 1, wherein R₅ is C1-C5 alkyl.

5. The method of claim 1, wherein R₆ is C1-C5 alkyl.

6. The method of claim 1, wherein the compound represented by formula (I) is a compound represented by the following formula (I-1):

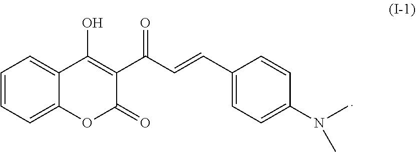 [SMILES: CN(C)c1ccc(/C=C/C(=O)c2c(O)c3ccccc3oc2=O)cc1]

7. The method of claim 1, wherein a concentration of the compound represented by the formula (I) is in a range from 0.0005 μM to 200 μM based on a total weight of the pharmaceutical composition.

8. The method of claim 1, wherein the protein aggregation disease is Alzheimer's disease or Parkinson's disease.

9. The method of claim 1, wherein the protein aggregation disease is Alzheimer's disease.

10. The method of claim 1, wherein the protein aggregation disease is Parkinson's disease.

11. A method for inhibiting a protein aggregation in a subject in need thereof, comprising:
administering a pharmaceutical composition comprising a compound represented by the following formula (I) to the subject, 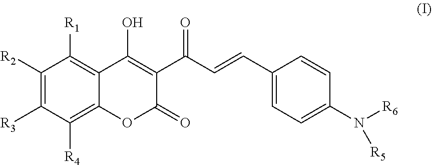
wherein each R₁, R₂, R₃, R₄, R₅ and R₆ is independently selected from the group consisting of H and alkyl.

12. The method of claim 11, wherein each R₁, R₂, R₃, R₄, R₅ and R₆ is independently selected from the group consisting of H and C1-C5 alkyl.

13. The method of claim 11, wherein each R₁, R₂, R₃, and R₄ is H.

14. The method of claim 11, wherein R₅ is C1-C5 alkyl.

15. The method of claim 11, wherein R₆ is C1-C5 alkyl.

16. The method of claim 11, wherein the compound represented by formula (I) is a compound represented by the following formula (I-1):

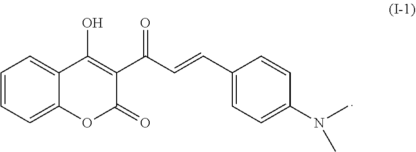 [SMILES: CN(C)c1ccc(/C=C/C(=O)c2c(O)c3ccccc3oc2=O)cc1]

17. The method of claim 11, wherein a concentration of the compound represented by the formula (I) is in a range from 0.0005 μM to 200 μM based on a total weight of the pharmaceutical composition.

18. The method of claim 11, wherein the protein aggregation is β-amyloid aggregation.

19. The method of claim 11, wherein the protein aggregation is tau protein aggregation.
